(19) United States
(12) Patent Application Publication
(10) Pub. No.: US 2007/0004727 A1
(43) Pub. Date: Jan. 4, 2007

(54) Use of compounds for the prevention of drug-induced cell toxicity

(75) Inventor: Anders Nykjaer, Risskov (DK)

Correspondence Address: BROWDY AND NEIMARK, P.L.L.C.;624 NINTH STREET, NW, SUITE 300, WASHINGTON, DC, 20001-5303, US

(73) Assignee: Reception ApS c/o Ostysk Innovation A/S, Arhus c (DK)

(21) Appl. No.: 10/550,488

(22) PCT Filed: Mar. 25, 2004
(86) PCT No.: PCT/DK04/00205

§ 371 Date: Aug. 21, 2006

(30) Foreign Application Priority Data
Mar. 26, 2003 (DK) PA200300459

(51) Int. Cl. A61K 31/7048; A61K 31/727; A61K 31/66; A61K 31/53; A61K 31/497; A61K 31/496; A61K 31/495; A61K 31/7034; A61K 31/13

(52) U.S. Cl. 514/241; 514/252.12; 514/252.14; 514/255.02; 514/255.03; 514/673; 514/565; 514/383; 564/511; 564/512; 514/28; 514/263.31; 514/263.32; 514/190; 514/200; 514/171; 514/35; 514/165; 514/210.09; 514/102; 514/56; 424/85.4; 424/85.2; 424/450; 514/29; 514/254.07; 514/355; 514/381; 514/214.01; 514/284; 514/282

(57) ABSTRACT

The present invention relates to the use of compounds for the manufacture of a medicament for the prophylaxis and/or treatment of induced cell toxicity, such as nephrotoxicity and ototoxicity, in particular where the cell toxicity is induced by a medical treatment. In a preferred embodiment the compounds have at least two nitrogen atoms, more preferably at least two amino groups. The compounds according to the invention are capable of blocking binding of cell toxic compounds to the megalin receptor, and thereby inhibiting uptake of the cell toxic compounds into cells. The invention further relates to novel compounds for use in said treatment, as well as a method for reducing the cell toxicity of cell toxic compounds.

### FIELD OF INVENTION

The present invention relates to the technical field of cell toxicity treatment and discloses compounds and combination medicaments for use in such treatment.

### BACKGROUND OF INVENTION

The invention relates to compounds for the prevention of organ damage, in particular organ damage of the kidneys and the inner ear induced by the administration of therapeutic agents.

Several classes of drugs in clinical use are toxic to tissues like the kidney and inner ear. Prominent drugs in this category are cisplatin, ifosfomide, ciclosporin, amphotericin B, valproate, polymyxin B and therapeutic antibodies. However, of particular importance are aminoglycosides which are among the most important antibiotics for the treatment of severe bacterial infections. They are the preferred agents against a number of Gram-negative bacteria. At present the market share of aminoglycosides in the field of anti-infectious agents is rapidly increasing. Above all, this is due to a general increase in the occurrence of pathological strains resistant to other classes of antibiotics.

The main obstacle in the clinical use of the above drugs is their severe oto- and nephrotoxic (ear and kidney) side effects, which may lead to complete loss of hearing and to renal failure in the long term. The use of these drugs is thus not only associated with a high risk but also entails high costs for drug monitoring and diagnosis. Their use is therefore restricted to incidences of the most severe infections in the industrial countries. In the developing countries, where aminoglycosides are used more frequently because of their low production costs, aminoglycosides account for 70% of all cases of acquired deafness.

The underlying mechanisms causing toxicity are not understood. So far it is known that the drugs bind to the surface of cells in the kidneys and the inner ear and are taken up into the cells through unknown mechanisms. As the drugs are poorly degradable in the cells, they accumulate intracellularly leading to the destruction of cell structures and thus to renal damage and hearing loss. Various surface structures or receptors have been held responsible for the binding and uptake of the antibiotics.

Moestrup et al. suggested that megalin, a surface receptor of the kidneys, is responsible for the uptake of antibiotics (Moestrup et al., J. Clin. Invest. 96, 1404-1413, 1995). Megalin is a 600 kDa endocytosis receptor of the low-density lipoprotein (LDL) receptor gene family. Megalin is a multifunctional clearance receptor that binds and internalises a number of macromolecules. The sequence for the receptor megalin is shown as: cDNA: U33837; gene: NT_{—}002176.

Another receptor believed to be involved in antibiotic interaction is cubilin. Cubilin is a 460 kDa membrane-associated protein colocalizing with megalin, that may facilitate the endocytic process by sequestering the antibiotic/therapeutic agent on the cellular surface before megalin-mediated internalization of the cubilin-bound ligand. In other words, the therapeutic agent may bind to cubilin as well as directly to megalin. Cubilin, however, appears not to be able to mediate endocytosis on its own but physically associates with megalin and internalizes in a complex with this receptor. The sequence for the receptor cubilin is shown as: cDNA: XM_{—}011904; gene: NT_{—}008682 (Homo sapiens chromosome 10 working draft sequence segment).

Several strategies for preventing toxic side effects of aminoglycosides have been developed. Examples are the development of novel aminoglycosides having fewer side effects (see amikacin, a semi-synthetic derivative of kanamycin (Begg, E. J. & Barclay, M. L. Br. J. Clin. Pharmac. 39, 597-603, 1995)), and the simultaneous administration of aminoglycosides with other compounds, such as neutrophin-3 (Ernfors, P., Duan, M. L., ElShamy, W. M. & Canlon, B. Nat Med. 2, 463-467 (1996)), nitrendipine (Lee, S. M., Pattison, M. E. & Michael, U. F. J. Cardiovasc. Pharmacol. 9, S65-S69 (1997)), Pyrola rotundifolia (Xuan, W. & Dong, M. Ann. Otol. Rhinol. Laryngol. 104, 374-380 (1995)) or antioxidants (Schacht, J. Head and Neck Surgery 118, 674-677 (1998)).

In Jones et al. (Jones, M. M., Basinger, M. A., and Holscher, M. A.; Fundamental and applied toxicology, 18, 181-188 (1992)) the control of nephrotoxicity of cisplatin by clinically using sulphur-containing compounds is described. Jones et al. disclose how sulphur-containing compounds bind to hydrolytic products derived from the platin part of cisplatin and thereby reduces the nephrotoxic side effect of cisplatin. The article does not disclose cell toxicity reducing compounds or medicaments capable of binding to a receptor cubilin and/or a receptor megalin and/or a co-receptor of megalin and cubilin.

The present invention presents compounds having an improved cell toxicity reducing effect when used alone or in combination with a therapeutic agent causing said cell toxicity.

### SUMMARY OF INVENTION

Thus the invention discloses the use of a compound comprising a structure of the general formula (I)
—N′—X—Y—N″— (I)
wherein X represents one atom and Y represents at least one atom and X and Y may individually be substituted at least once, or a pharmaceutically acceptable addition salt or hydrate thereof,
for the manufacture of a medicament for the prophylaxis and/or treatment of induced cell toxicity,
with the proviso that the compound is not biotin when the agent inducing cell toxicity is cisplatin.

Further, the invention concerns the use of a compound comprising a structure of the general formula (IV) 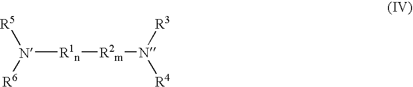
wherein
each R¹ and each R² independently are selected from C, S, N, O, optionally substituted with C, S, N, O, OH, hydrogen, alkyl, alkenyl, alkynyl, phenyl, benzyl, amine (NH), halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, optionally further substituted one or more times with C, S, N, O, OH, phenyl, amine (NH), halogen, substituted lower alkyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, halogen, trifluoromethyl, cyano, amino, or nitro, and wherein
ₘ is an integer of from 1 to 8,
ₙ is an integer of from 1 to 8,
N′ and N″ are nitrogen,
R³, R⁴, R⁵ and R⁶ are independently selected from C, S, N, O, OH, hydrogen, alkyl, alkenyl, alkynyl, phenyl, benzyl, amine (NH), halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, optionally further substituted one or more times with C, S, N, O, OH, phenyl, amine (NH), halogen, substituted lower alkyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, halogen, trifluoromethyl, cyano, amino, or nitro,
or one or more of R³, R⁴, R⁵ and R⁶ is a chemical bond,
or a pharmaceutically acceptable addition salt or hydrate thereof,
for the manufacture of a medicament for the prophylaxis and/or treatment of induced cell toxicity.

When any variable occurs more than once in any constituent, each definition is independent.

In another aspect of the invention a combination medicament comprising a compound of the invention and a therapeutic agent for simultaneous, separate or se-quential use in cell toxicity therapy, said cell presenting a receptor megalin and/or a receptor cubilin and/or a co-receptor of megalin and cubilin is disclosed.

The invention also discloses the use of a compound being a moiety of gentamicin for the prophylaxis and/or treatment of induced cell toxicity, wherein said cell presents the receptor megalin and/or the receptor cubilin as well as the compounds called Garoseamine, 2-deoxystreptamine and Purpurosamine for the prophylaxis and/or treatment of induced cell toxicity.

The present invention is also concerned with a compound having the general formula (Aq-X)ₚ (VII) wherein

A is independently or in combination selected from formula (I) and/or formula (II) and/or formula (III) and/or formula (IV) and/or formula (V) and/or formula (VI), Garoseamine, Purpurosamine, and 2-deoxystreptamine, and wherein

X is a spacer, q is an integer of 1-150, p is an integer of 1-150, and its use for the prophylaxis and/or treatment of induced cell toxicity.

In a further embodiment the invention relates to a method for reducing cell toxicity of at least one cell toxic compound.

The invention may also be used when administering cell toxic compounds locally, such as by inhalation, and wherein systemic uptake is not desired. Since the uptake of the cell toxic compounds is believed to be due to binding to a megalin receptor, blocking said receptor may prevent systemic uptake. In particular when treating lung and bronchial infections, it may be advantageous to co-administer a compound according to the present invention together with the antibiotic compound, in order to reduce systemic uptake.

Accordingly, the invention further relates to use of a compound according to the invention for preparation of a medicament for inhibiting systemic uptake of a cell toxic compound. The compound according to the invention is preferably administered locally in the same administration form as the cell toxic compound.

### DESCRIPTION OF DRAWINGS

FIG. 1: shows examples of gentamicin-inhibitor compounds.

FIG. 2: shows possible moieties of gentamicin.

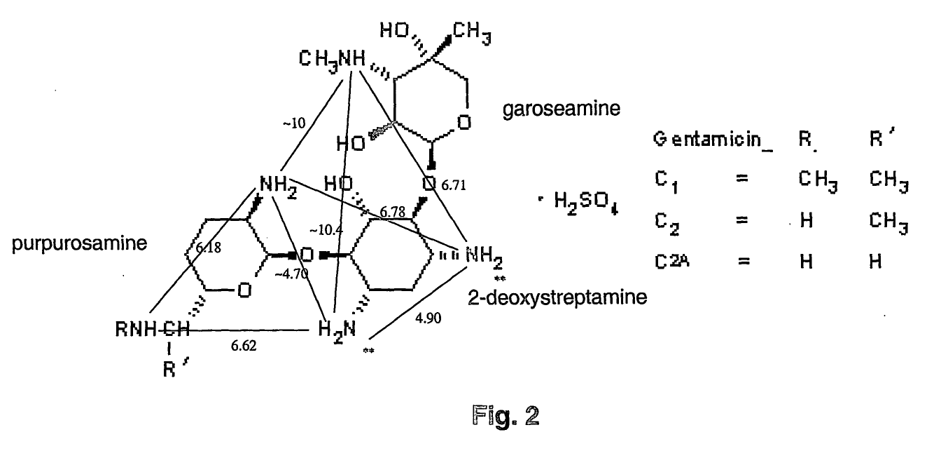

FIG. 3: shows the 3D structure of gentamicin.

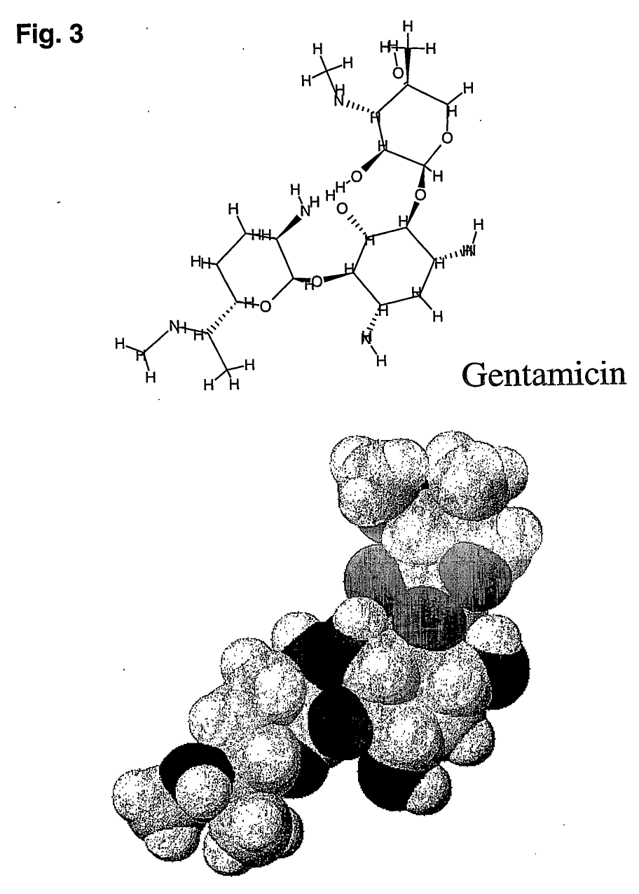

FIG. 4: shows the inhibition of the receptor megalin by selected inhibitors.

FIG. 5: shows the inhibition of the receptor megalin by selected inhibitors.

FIG. 6: shows the uptake of gentamicin in mouse kidneys following i.p. administration of 3 mg of selected compounds.

FIG. 7: shows selected gentamicin inhibitors.

### DETAILED DESCRIPTION OF THE INVENTION

Definitions:

Alkyl group: the term “alkyl group” means a saturated linear or branched hydrocarbon group including, for example, methyl, ethyl, isopropyl, t-butyl, heptyl, dodecyl, octadecyl, amyl, 2-ethylhexyl, and the like.

Substituted lower alkyl means a lower alkyl having one to three substituents selected from the group consisting of hydroxyl, alkoxy, amino, amido, carboxyl, acyl, halogen, cyano, nitro and thiol.

Heterocyclyl means a monovalent saturated cyclic radical, consisting of one to two rings, of three to eight atoms per ring, incorporating one or two ring heteroatoms (chosen from N, O or S(O)₀₋₂, and which can optionally be substituted with one or two substituents selected from the group consisting of hydroxyl, oxo, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminofarbonyl, arylaminocarbonyl, alkylcarbonylamino, or arylcarbonylamino.

Heteroaryl means a monovalent aromatic cyclic radical having one to three rings, of four to eight atoms per ring, incorporating one or two heteroatoms (chosen from nitrogen, oxygen, or sulfur) within the ring which can optionally be substituted with one or two substituents selected from the group consisting of hydroxy, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonlamino and arylcarbonylamino.

Cycloalkyl means a monovalent saturated carbocyclic radical consisting of one or two rings, of three to eight carbons per ring, which can optionally be substituted with one or two substituents selected from the group consisting of hydroxy, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino and arylcarbonylamino.

Induced cell toxicity means a therapeutic agent capable of inducing a toxic response in the cell being exposed to said therapeutic agent.

Cell toxicity: an agent is defined as toxic when it is directly capable of causing cell death.

Cell death: in the present context cell death is defined in various ways and covers a cell which has lost all its functions, a cell which has lost a special function, such as hormone synthesis, or a cell which has a reduced capability for further division.

Cationic group: A chemical group capable of functioning as a proton donor when a compound comprising the chemical group is dissolved in a solvent, preferably when dissolved in water.

Moieties of a particular compound cover group(s) or part(s) of said particular compound.

Spacer in the present context refers to the atoms directly linking the monomers of formula (I), (II), (III), (IV), (V) or (VI). The spacer may also directly link the compounds of formula (I), (II), (III), (IV), (V) and/or (VI) to a therapeutic agent as described by the present combination medicament.

Form a ring means that the atoms mentioned are connected through a bond when the ring structure is formed.

Therapeutic agent is used synonymously with a medicament, unless otherwise stated.

The principle of the present invention is to reduce the side effects caused by therapeutic agents, in particular kidney and inner ear damage. The toxicity of the therapeutic agents is possibly due to the accumulation of the therapeutic agent in cells in the organs in question. Thus, the invention is focused on the inhibition of the accumulation in the cells of the therapeutic agent.

Accordingly, the present invention relates to the use of novel compounds capable of inhibiting the intracellular accumulation. This may for example be done by inhibiting the binding of the therapeutic agent to the receptor megalin by either blocking a sufficient amount of binding sites on the receptor megalin and/or blocking the therapeutic agent so that it maintains the normal therapeutic effect but is inhibited from binding to the receptor.

By the present invention the novel use of compounds acting as antagonists for use in the manufacture of medicaments for the prophylaxis and/or treatment of induced cell toxicity is disclosed. Further, novel compounds and moieties of the known antibiotic gentamicin and their therapeutic use as cell toxicity reducing agent are described.

Novel Use of Compounds

It is within the scope of the invention that the compounds of the invention may bind to any given receptor involved in induced cell toxicity in order to inhibit binding and optionally uptake of the therapeutic agent into the cell.

The compound is preferably either capable of binding to a sufficient amount of binding sites on the receptor(s) or of binding to the receptor and sterically hindering the binding of the therapeutic agent.

Accordingly, in the broadest aspect the present invention concerns the use of a compound comprising a structure of the general formula (I)
—N′—X—Y—N″— (I)
wherein, X represents one atom and Y represents at least one atom and X and Y may individually be substituted at least once, and
N′ and N″ are nitrogen, sulphur or phosphor (in a preferred embodiment, at least one of N′ or N″ is nitrogen; more preferably both N′ and N″ are nitrogen),
for the manufacture of a medicament for the prophylaxis and/or treatment of induced cell toxicity,
with the proviso that the compound is not biotin.

The number of atoms between N′ and N″ is at least two as mentioned above. However, the number of atoms between N′ and N″ may be at least three, such as at least four, for example at least five, such as at least six, for example at least seven, such as at least eight.

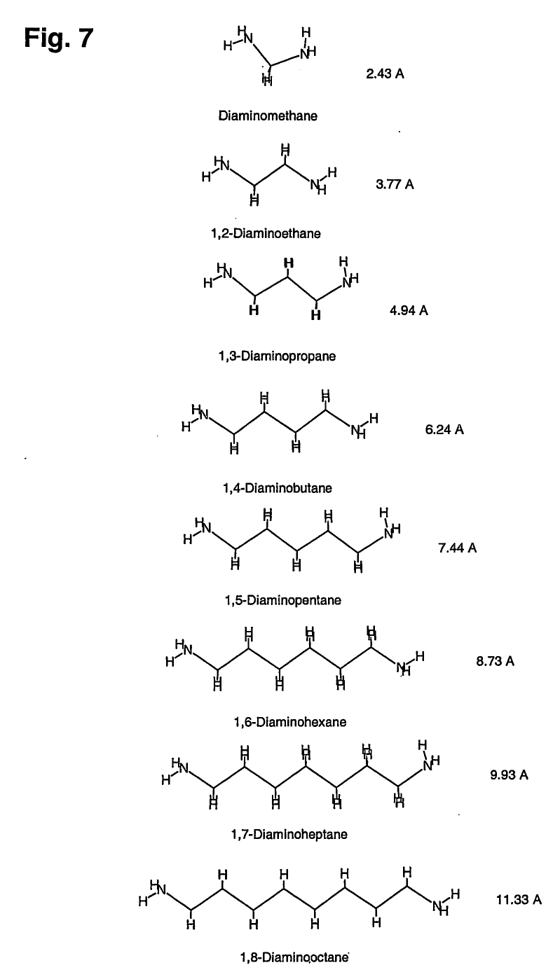

However, in a preferred embodiment of the invention the number of atoms between N′ and N″ is an even number, such as two atoms, for example four atoms, such as six atoms, for example eight atoms. Compounds having an even number of atoms between N′ and N″ are illustrated by the invention in FIG. 7, where for example 1,6-diaminohexane has six atoms between N′ and N″. According to the invention it has been found that compounds having an even number of atoms between N′ and N″ have particular promise as induced toxicity inhibitors. Thus, in a preferred embodiment, two atoms separate N′ and N″. In another embodiment, four atoms separate N′ and N″. In yet another embodiment, six atoms separate N′ and N″.

In one embodiment of the invention the use of a compound comprising a structure of the general formula (II)
—N′—C—C—N″— (II)
wherein N′ and N″ are nitrogen,
or a pharmaceutically acceptable addition salt or hydrate thereof,
for the manufacture of a medicament for the prophylaxis and/or treatment of induced cell toxicity is disclosed.

In another embodiment, the compound comprises a structure of the general formula (III)
—N′—C—N—C—N″— (III)
wherein, N′ and N″ are nitrogen,
or a pharmaceutically acceptable addition salt or hydrate thereof,
for the manufacture of a medicament for the prophylaxis and/or treatment of induced cell toxicity.

In one embodiment, the invention relates to the use of a compound comprising a structure of the general formula (IV) 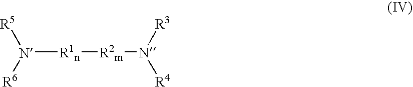
wherein
each R¹ and each R² independently are selected from C, S, N, O, optionally substituted with C, S, N, O, OH, hydrogen, alkyl, alkenyl, alkynyl, phenyl, benzyl, amine (NH), halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, optionally further substituted one or more times with C, S, N, O, OH, phenyl, amine (NH), halogen, substituted lower alkyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, halogen, trifluoromethyl, cyano, amino, or nitro, and wherein
ₘ is an integer of from 1 to 8,
ₙ is an integer of from 1 to 8,
N′ and N″ are nitrogen,

R³, R⁴, R¹ and R⁶ are independently selected from C, S, N, O, OH, hydrogen, alkyl, alkenyl, alkynyl, phenyl, benzyl, amine (NH), halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, optionally further substituted one or more times with C, S, N, O, OH, phenyl, amine (NH), halogen, substituted lower alkyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, halogen, trifluoromethyl, cyano, amino, or nitro,

or one or more of R³, R⁴, R⁵ and R⁶ is a chemical bond,

or a pharmaceutically acceptable addition salt or hydrate thereof,

for the manufacture of a medicament for the prophylaxis and/or treatment of induced cell toxicity.

In a preferred embodiment, the compound consists of the structure of the general formula (IV).

Provisos

The above mentioned compounds of the invention may, however, not be the compound biotin having the formula shown below when the therapeutic agent causing cell damage is cisplatin: 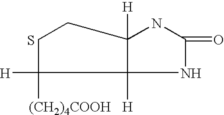
Structure of the Compounds
Radicals/Ring Formations

The present invention is concerned with toxicity inhibiting compounds having various structures. Such structures may be linear structures or cyclic structures. Regardless of the specific structure of the present compound, the atoms of the compounds must be positioned in a way that allows for the compound to exert its inhibitory effect. Such inhibitory effect may result from the antagonistic binding of the compound to a receptor involved in cell toxicity and/or result from the binding of the compound to a therapeutic agent resulting in the prevention of a functional binding between the therapeutic agent and its receptor. By “functional binding” is meant a binding between the therapeutic agent and its corresponding receptor resulting in a cell toxic response.

The linear or cyclic structures of the present invention as mentioned above are determined by the nature of the radicals as mentioned herein.

Accordingly, in one embodiment of the invention the radicals R⁶ and R² form a ring or the radicals R⁵ and R² form a ring.

There are other embodiments in which radicals form a cyclic structure. In one such embodiment, R³ and R¹ form a ring or R⁴ and R¹ form a ring; an example hereof is shown as general formula (V). In another embodiment it is the radicals R⁴ and R⁶ forming a ring; an example hereof is shown as general formula (VI).

It is also within the scope of the invention to provide compounds comprising a cyclic structure between a radical and N′ or N″. Thus, in a preferred embodiment R⁴ and N′ form a ring.

In another preferred embodiment the radical R⁶ forms a ring structure with N″.

The term “ring” is used synonymously with the term “cyclic”.

The above cyclic structures of the compounds may have a variety of ring members depending on the nature of the chemical group(s) involved in the cyclic structure. Therefore in one embodiment of the invention the ring structure of the compound is 5-membered. In another embodiment of the invention the ring structure is 6-membered. Furthermore, in yet another embodiment of the invention the ring structure of the compound is 7-membered. Preferably, the ring structure is 6-membered.

Charge

The compounds according to the invention are capable of donating at least one proton, more preferably at least two protons because the structure of such compounds allows cell toxicity inhibition to be manifested. Accordingly, the compounds preferably have at least one amino group capable of functioning as a proton donor. More preferably, the compounds have two amino groups capable of functioning as a proton donor.

Accordingly, in a preferred embodiment of the invention, at least one of R³, R⁴, R⁵ and R⁶ is H.

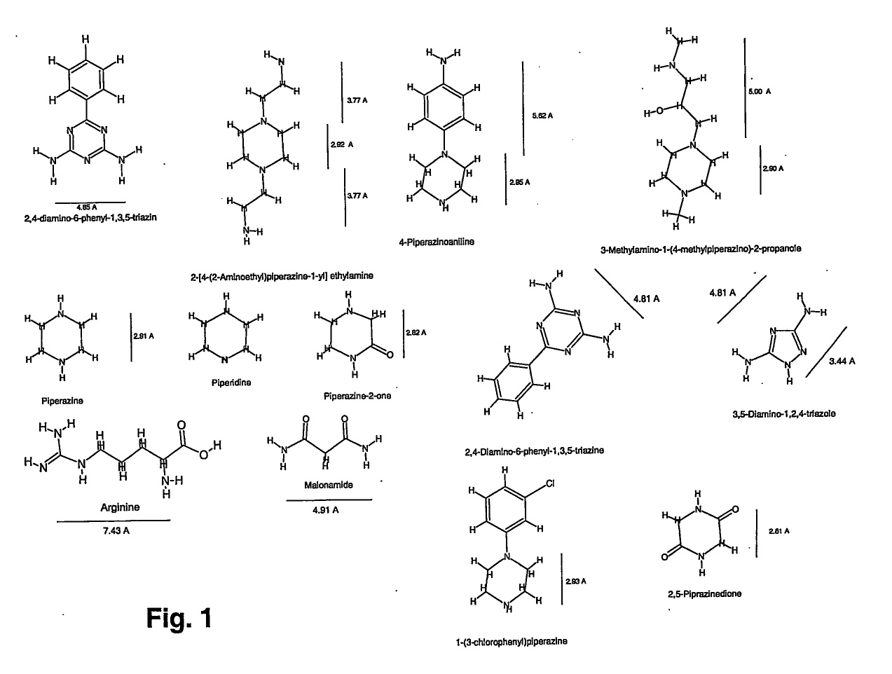

In a more preferred embodiment of the invention, R³ and R⁵ are H. For example a compound wherein the two radicals are both represented by a hydrogen atom is illustrated by the present compound piperazine (see FIG. 1).

Polybasic Charge Distribution

According to the invention it has been found that compounds comprising a polybasic charge distribution are particularly useful as inhibitors of induced cell toxicity.

Thus, as discussed above, in a preferred embodiment of the invention the present compound has at least 1, such as 2 positive charges in solution, such as at least 2 positive charges, such as 2 positive charges.

By selecting the positive charges within an interval of from 1 to 300 it is normally possible to block a sufficient number of binding sites on the receptor responsible for mediating induced toxicity, such as the receptor megalin and/or the receptor cubilin and/or a co-receptor of megalin and cubilin. This results in the inhibition of uptake of the therapeutic agent responsible for inducing cell toxicity.

In a preferred embodiment the compound exhibits at least 3 positive charges, such as at least 5 positive charges, such as at least 10 positive charges.

In one embodiment, it is preferred that the polybasic charge distribution has the same charge distribution as the cell toxicity inducing therapeutic agent.

In the case of the receptor cubilin and the receptor megalin, a plausible explanation why a polybasic charge distribution is preferred may be that the binding of the aminoglycoside gentamicin to the receptor(s) is not necessarily dependent on the native conformation of the receptor, since reduction of disulfide-bridges does not significantly interfere with ligand binding. Moreover, the addition of EDTA, which depletes the presence of calcium and affects receptor stability, does not abolish binding. This indicates that the interaction between receptor and gentamicin may depend on simple ionic interactions rather than the overall confirmation of the receptor.

General Formulae (V) and (VI)

In one embodiment, the present invention also relates to the use of compounds having a structure of the general formula (V) 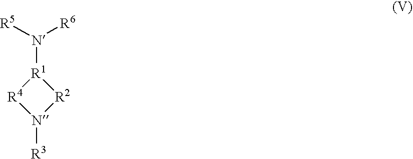
or the general formula (VI) 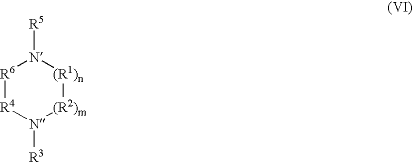
wherein
N′ and N″, ₘ, ₙ, R¹, R², R³, R⁴, R⁵ and R⁶ are as defined above for the formula (IV), or a pharmaceutically acceptable addition salt or hydrate thereof, for the manufacture of a medicament for the prophylaxis and/or treatment of induced cell toxicity.

For the compounds of the invention ₘ and ₙ are preferably an integer of from 1-7, such as from 1-6, for example an integer of from 1-5, such as from 1-4, such as from 1-3. Most preferably, both ₘ and ₙ are 1.

Chemical Bond

The radicals of the present invention may be represented by the chemical group(s) already disclosed above. However, it is also within the present invention to provide compounds comprising radicals represented by a chemical bond. In embodiments where one or more of the radicals are chemical bonds, compounds comprising structures, such as cyclic structures mentioned above are obtained.

Accordingly, in one embodiment at least three of R³, R⁴, R⁵ and R⁶ are a chemical bond.

In another embodiment at least two of R³, R⁴, R⁵ and R⁶ are a chemical bond.

Further, in another embodiment at least one of R³, R⁴, R⁵ and R⁶ is a chemical bond.

The chemical bond of the invention may be any chemical bond, such as a covalent bond.

To illustrate the principle of the radicals being chemical bonds imagine that R⁶ of the above formula (VI) is representing a chemical bond. In this particular embodiment the 6-membered ring structure of formula (VI) would change into a compound comprising a 5-membered ring structure. At the same token other compound structures may change according to the radical being a chemical bond instead of a chemical group.

Binding to the Receptor Cubilin and/or Megalin

The compounds of the present invention are all capable of binding to the receptor megalin and/or the receptor cubilin and/or a co-receptor of megalin and cubilin. Additionally the compounds according to the invention are capable of binding to the therapeutic agent. In case of binding to the receptor(s) it is of importance that the binding is effective in respect of blocking the binding of the therapeutic agent to the receptor. The receptor megalin for example comprises 50-150 binding sites for the therapeutic agent gentamicin and it is important for the effectiveness of the use of this invention that the compound is capable of inhibiting an effective amount of these binding sites.

Without being bound by theory the advantage of the present compound's ability of binding to the receptor cubilin and/or receptor megalin is the finding that these receptors are involved in aminoglycoside induced cell toxicity in the kidneys and the ear. Using the present compounds has an inhibitory effect on cell toxicity, such as nephrotoxicity and ototoxicity.

The compounds according to the invention may bind either the receptor megalin in order to inhibit endocytosis or the receptor cubilin in order to reduce its sequestering and thereby inhibiting or reducing endocytosis.

Co-Receptor

It is also within the scope of the invention that the compound may bind to a co-receptor of megalin and cubilin.

Distance Between N Atoms

It has been found that the distance between the N′ and N″ atoms of the compounds (see all the present formulas) plays a role in the effectiveness of the present inhibitor.

Thus, in an important aspect of the invention the distance between the N′ and N″ atoms of the compounds of the invention is between 2.0 and 9.0 Ångstrøm, and in a preferred embodiment the distance between N′ and N″ is preferably between 2.4 and 8.5 Ångstrøm, such as between 2.5 and 3.5 Å, such as between 2.7 and 3.3 Å, such as about 2.9 Å, or such as between 7.5 and 8.5 Å, such as between 8.0 and 8.5 Å. When at least one N atom is part of a ring structure, the distance is preferably between 2.5 and 3.5 Å. When the compound is a linear structure, the distance is preferably between 7.5 and 9.0 Å.

R¹/R²

The radicals R¹ and R² may, according to the invention, represent chemical group(s) as defined in the present formulas. There are, however, compounds in which the specific nature of the radicals R¹ and R² has proven to be particularly successful. In one such preferred embodiment at least one of the radicals R¹ or R² is represented by a C.

In a more preferred embodiment of the invention the radicals R¹ and R² are both represented by C.

It is also envisioned that the present compound in a further embodiment comprises radicals wherein at least one of R¹ or R² is S. In a more preferred embodiment the radicals R¹ and R² are both represented by S.

Furthermore, the present compound may in a further embodiment comprise at least one of R¹ and R² being represented by N, such as both the radicals R¹ and R² being represented by N.

The present compound may also comprise radicals wherein at least one of R¹ and R² is O. In a more preferred embodiment, however, the radicals R¹ and R² are both represented by O.

Compounds

Examples of compounds according to invention are selected from diaminomethane, 1,2-diaminoethane, 1,3-diaminopropane, 1,4-diaminobutane, 1,5-diaminopentane, 1,6-diaminohexane, 1,7-diaminoheptane, 1,8-diaminooctane, 3-methylamino-1-(4-methylpiperazino)-2-propanole, 4-piperazinoaniline, 1-(3-chlorophenyl)piperazine diHCl (m-CPP), piperazin-2-one-HCl, 2-[4-(2-aminoethyl)piperazin-1-yl]ethylamine, piperazine anhydrous, 2,4-diamino-6-phenyl-1,3,5-triazine, 3,5-diamino-1,2,4-triazole, melonamide, arginine-HCl, piperidine, 2,5-piperazinedione, piperazine, piperazin-2-one-HCl, 1-(2-pyrimidyl)-piperazine dihydrochloride

Examples of preferred compounds are selected from 2-[4-(2-aminoethyl)piperazin-1-yl]ethylamine, 3-methylamino-1-(4-methylpiperazino)-2-propanole, and piperazine, piperazin-2-one, or a pharmaceutically acceptable addition salt or hydrate thereof.

In a more preferred embodiment the compound is piperazine or a pharmaceutically acceptable addition salt or hydrate thereof.

In another embodiment of the invention the compound may be selected from 1,2-diaminoethane, 1,3-diaminopropane, 1,4-diaminobutane, 1,5-diaminopentane, 1,6-diaminohexane, 1,7-diaminoheptane, 1,8-diaminooctane or a pharmaceutically acceptable addition salt or hydrate thereof.

In yet a further embodiment the compound may be selected from 1,7-diaminoheptane, 1,2-diaminoethane, 1,4-diaminobutane, 1,6-diaminohexane, 1,5-diaminopentane or a pharmaceutically acceptable addition salt or hydrate thereof.

In a most preferred embodiment the compound is 1,6-diaminohexane or a pharmaceutically acceptable addition salt or hydrate thereof.

A pharmaceutically acceptable salt means any salt of the compounds mentioned. In particular, it means a pharmaceutically acceptable acid addition salt. Pharmaceutically acceptable acid addition salts of the compounds include salts derived from nontoxic inorganic acids such as hydrochloric, nitric, phosphoric, sulfuric, hydrobromic, hydriodic, hydrofluoric, phosphorous and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, di-hydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like.

Use of Gentamicin Derived Compounds

As previously mentioned it is known that gentamicin binds to the receptor cubilin and the receptor megalin. It is within the present scope of the invention to describe the use of compounds derived from gentamicin against induced cell toxicity.

Thus, in one aspect the invention concerns the use of a compound being a moiety of gentamicin for the prophylaxis and/or treatment of induced cell toxicity, wherein the cell presents the receptor megalin and/or the receptor cubilin and/or co-receptors to megalin or cubilin.

The compounds being derived from the aminoglycoside gentamicin conventionally used as an antibiotic have surprisingly been found to have inhibitory effects on the cell toxicity induced by gentamicin. Without being bound by theory it is envisioned that the gentamicin-derived compounds exert their inhibitory effect through mechanisms as described earlier in the present text.

According to the invention in one embodiment one of the gentamicin-derived compounds is a moiety called Garoseamine. In another embodiment the gentamicin-derived compound is the moiety Purpurosamine. In yet another embodiment of the invention the gentamicin-derived compound is the moiety 2-deoxystreptamine.

Thus, in one embodiment the compound to be used in the prophylaxis and/or treatment of induced cell toxicity, wherein said cell presents the receptor megalin and/or the receptor cubilin and/or a co-receptor of megalin and cubilin, is Garoseamine.

In another embodiment the compound to be used in the prophylaxis and/or treatment of induced cell toxicity, wherein said cell presents the receptor megalin and/or the receptor cubilin and/or a co-receptor of megalin and cubilin, is Purpurosamine.

In yet a further embodiment the compound to be used in the prophylaxis and/or treatment of induced cell toxicity, wherein said cell presents the receptor megalin and/or the receptor cubilin and/or a co-receptor of megalin and cubilin, is 2-deoxystreptamine.

Novel Polymers

The invention further presents novel compounds to be used to reduce induced cell toxicity.

Thus, the invention presents a compound having the general formula
(A_{q}-X)ₚ (VII)
wherein

A is independently or in combination selected from formula (I) and/or formula (II) and/or formula (III) and/or formula (IV) and/or formula (V) and/or formula (VI), Garoseamine, Purpurosamine, and 2-deoxystreptamine, and wherein

X is a spacer,

q is an integer of 1-150,

p is an integer of 1-150.

According to the invention q may be an integer of 1-100, 2-150, 2-100, 2-50, 2-10 or 5-95, for example 10-90, such as 15-90, for example 20-85, such as 25-80, for example 30-75, such as 35-70, for example 40-65, such as 45-60, for example 50-55.

Further, p of the formula may be an integer of 1-100, 2-150, 2-100, 2-50, 2-10 or 5-95, for example 10-90, such as 15-90, for example 20-85, such as 25-80, for example 30-75, such as 35-70, for example 40-65, such as 45-60, for example 50-55.

In one embodiment, A is independently or in combination selected from formula (I) and/or formula (II) and/or formula (III) and/or formula (IV) and/or formula (V) and/or formula (VI).

It is envisioned that one or more monomer(s) of the compound of formula (I) may be linked by a spacer to one or more monomer(s) of the compound of formula (II) or to one or more monomer(s) of the compound of formula (III) or to one or more monomer(s) of the compound of formula (IV) or to one or more monomer(s) of the compound of formula (V) or to one or more monomer(s) of the compound of formula (VI).

In another embodiment one or more monomer(s) of the compound of formula (I) may be linked by a spacer to one or more monomer(s) of the compound of formula (I). Likewise, the compounds of formula (II), (III), (IV), (V) and (VI), respectively, may also be linked to one or more monomers of the compounds of formula (II), (III), (IV), (V) and (VI), respectively.

In a further embodiment one or more monomer(s) of the compound of formula (I) may be linked by a spacer to one or more monomer(s) of the compound of formula (II) and/or one or more monomer(s) of the compounds of the formula (III), (IV), (V) an/or (VI), respectively, and further be linked by a spacer to one or more monomer(s) of the compound of formula (III), (IV), (V) and/or (VI), respectively.

It is to be understood that the individual monomers of the above embodiments belonging to the same group formula (i.e. formula (I), (II), (III), (II), (III), (IV), (V) and (VI), respectively) are linked by spacers just like the individual monomers/polymers belonging to different group formulas are linked by spacers.

The combinations of the present compounds may be illustrated by the following examples:

formula (I)-formula (I)-spacer-formula (I)-formula (I)

formula (I)-spacer-formula (I)-formula (I)-formula (I)

formula (I)-spacer-formula (I)-spacer-formula (I)-formula (I)

formula (I)-formula (II)-spacer-formula (I)-formula (I)

formula (I)-spacer-formula (I)-formula (II)-formula (III)

formula (I)-spacer-formula (I)-spacer-formula (I)-formula (I)

Furthermore, A may be independently or in combination selected from Garoseamine, Purpurosamine and/or 2-deoxystreptamine.

The above combinations are merely non-limiting illustrations of the possible combinations possible within the scope of the present invention.

Spacer

According to one embodiment of the invention the spacer is a covalent bond.

In a preferred embodiment the spacer consists of from 2-12 atoms, such as C-atoms, such as from 4-10 atoms, such as C-atoms, preferably from 6-8 atoms, such as C-atoms.

The invention further relates to a method for reducing cell toxicity of a therapeutic agent comprising at least one cell toxic compound, said method comprising reducing the number of cationic groups from said at least one cell toxic compound. In particular the method relates to reducing nephrotoxicity or ototoxicity. As discussed above, a variety of therapeutic agents are nephrotoxic or ototoxic thereby limiting the use of the therapeutic agents. The present invention offers a method for reducing said toxicity. It is preferred that the number of cationic groups is reduced by at least one group, more preferably at least two cationic groups. The rationale behind the method is to reduce or even eliminate the possibility that the compound from binding to megalin receptors, and thereby become taken up into the cells.

The cationic group is preferably an amino group.

The reduction of the number of cationic groups may be provided by either substituting the cationic group with a non-cationic group or by introducing another group into the compound, thereby inhibiting donation of a proton from the cationic group.

Accordingly, in one embodiment the number of cationic groups is reduced by substituting at least one cationic group by another group, wherein said other group is not a cationic group. In particular the other group may be selected from H, OH, and lower alkyl (C1-C4).

In another embodiment the number of cationic groups is reduced by introducing another group into said compound, thereby inhibiting donation of a proton from the cationic group. In particular this is provided by introducing an amide group into the compound.

The reduction of cationic groups are preferably carried out during synthesis of the compound.

Examples of therapeutic agents to be reduced may be any of the following therapeutic agents:

acebutolol, acetazolamide, acyclovir, adefovir, albumin, alclofenac, alendronate, alitretinoin, altretamine, amikacin, amiloride, aminoglutethimide, amiodarone, amoxicillin, amoxicillin/clavulanic acid, amphotericin b, amphotericin b cholesteryl sulfate complex, amphotericin b lipid complex, amphotericin b liposome, amtolmetin, aniracetam, antacids, antazoline, anthraquinone laxatives, aprotinin, arginine, arsenic trioxide, asparaginase, aspirin, atenolol, atovaquone, auranofin, aurothioglucose, azacitidine, azathioprine, aziocillin, aztreonam, bacampicillin, bacitracin, bemetizide, benoxaprofen, betaine, bezafibrate, bismuth subcitrate, bleomycin, boric acid, brivudine, broxuridine, bumetanide, calcifediol, calcitriol, candesartan, candesartan/hydrochlorothiazide, canrenoate, capreomycin, captopril, carbenicillin, carboplatin, carmustine, carprofen, cefaclor, cefetamet, cefixime, cefinetazole, cefonicid, cefoperazone, cefoperazone/sulbactam, cefotaxime, cefotetan, cefoxitin, cefpirome, cefsulodin, ceftazidime, ceftezole, ceftibuten, ceftizoxime, ceftriaxone, cefuroxime, celecoxib, cephalexin, cephaloridine, cephalothin, cephapirin, cephradine, chlortetracycline, cidofovir, cilazapril, cimetidine, ciprofibrate, cisapride, cisplatin, clarithromycin, clodronate, clofibrate, cloxacillin, cocaine, codeine, colistin, corticotropin, cosyntropin, cotrimazine, cotrimoxazole, crisnatol, cyclacillin, cyclosporine, cysteamine, decitabine, delapril, delavirdine, demeclocycline, denileukin diftitox, desflurane, dextran, diatrizoate, diazoxide, dibekacin, diclofenac, diclofenac/misoprostol, dicloxacillin, dicumarol, didanosine, dihydroergotamine, dihydroergotamine/heparin, dihydrotachysterol, dirithromycin, dopamine, doxepin, doxorubicin hydrochloride liposome, doxycycline, edetate calcium disodium, edetate disodium, emetine, enflurane, enlimomab, epinephrine, epirubicin, ergocalciferol, ergotamine, erythromycin/sulfisoxazole, erythropoietins, ethanolamine oleate, ethyl chloride, etidronate, etodolac, etomidate, etretinate, everninomycin, fadrozole, fenbufen, fenofibrate, fenoprofen, fenoterol/ipratropium, flecainide, fleroxacin, floxacillin, flupirtine, flurbiprofen, formestane, foscarnet, fosinopril, fotemustine, framycetin, furosemide, gabexate, gadopentetate dimeglumine, gallium nitrate, gemcitabine, gemfibrozil, gentamicin, glycerin, gold sodium thiomalate, guanadrel, guanethidine, guar gum, halothane, hemiacidrin, hemin, hetastarch, homoharringtonine, hyaluronidase, hydrochlorothiazide, idarubicin, ifosfamide, imatinib, imipramine, indapamide, influenza vaccine, interferon alfa-2a, interferon alfa-2b, interferon beta, natural, interferon beta-1b, interferon gamma, interleukin-3, interleukin-4, interleukin-6, iobenguane I-131, iodixanol, iohexol, iopamidol, iopanoic acid, iopentol, iopromide, iotrolan, ioversol, ioxaglate, ioxilan, ioxithalamate, irinotecan, irofulven, isepamicin, isoflurane, isoniazid, isoxicam, kanamycin, ketamine, ketoconazole, ketoprofen, ketorolac, lenograstim, levofloxacin, lincomycin, liposomal nystatin, lisinopril, lithium, lobaplatin, lomustine, lonidamine, lornoxicam, losartan, loxapine, lymphocyte immune globulin, mannitol, mebendazole, mefenamic acid, meglumine antimoniate, melarsoprol, meropenem, mesna, metaraminol, methacycline, methicillin, methimazole, methocarbamol, methotrexate, methoxamine, metrizamide, metronidazole, meziocillin, milrinone, miltefosine, minocycline, minoxidil, mitoguazone, mitolactol, mitomycin, mitotane, molindone, morniflumate, morphine, moxalactam, muromonab-CD3, nabumetone, nafcillin, naproxen, nedaplatin, neomycin, netilmicin, niclosamide, nifedipine, niflumic acid, nifurtimox, nisoldipine, nitroprusside, norepinephrine, norfloxacin, ofloxacin, olsalazine, oxaliplatin, oxandrolone, oxaprozin, oxolinic acid, oxytetracycline, paclitaxel, pamidronate, paramethadione, paromomycin, pefloxacin, pemetrexed, pemirolast, penicillin G, pentamidine, pentostatin, peplomycin, perindopril, phenazopyridine, phenindione, phenobarbital, phenylbutazone, phenylpropanolamine, phenyloin, phosphates, piperacillin, pirarubicin, piretanide, piroxicam, plicamycin, poloxamer-188, polymyxin B, potassium perchlorate, praziquantel, proglumetacin, propylthiouracil, pyrimethamine/sulfadoxine, quinagolide, quinapril, quinine, rabbit antithymocyte globulin, raltitrexed, ranitidine, ranpirnase, recombinant human hemoglobin, rifampin, ritodrine, ritonavir, rofecoxib, rolitetracycline, rufloxacin, salsalate, sevoflurane, silver nitrate, silver sulfadiazine, simvastatin, sodium cellulose phosphate, sodium chloride, sodium fluoride, sodium stibogluconate, spironolactone, streptokinase, streptomycin, streptozocin, sulfamethoxazole, sulfasalazine, sulfinpyrazone, sulfisoxazole, sulindac, sulprostone, sultamicillin, suprofen, tacrolimus, tasonermin, teicoplanin, temafloxacin, teniposide, tenoxicam, tetracycline, thiopental, tiaprofenic acid, ticarcillin, ticrynafen, tiludronate, tiopronin, tobramycin, tocainide, tolazoline, tolmetin, torsemide, tramadol, triamterene, trimethadione, trimethaphan, trimethoprim, trimetrexate, trimipramine, troglitazone, tromethamine, typhoid vaccine, valsartan, vancomycin, zolimomab aritox, zomepirac, zopiclone.

Preferably, the therapeutic agent is selected from the group consisting of acyclovir, albumin, amikacin, amoxicillin, amoxicillin/clavulanic acid, amphotericin B, amphotericin B cholesteryl sulfate complex, amphotericin B lipid complex, amphotericin B liposome, aprotinin, arsenic trioxide, azathioprine, aziocillin, bacitracin, bismuth subcitrate, bleomycin, brivudine, calcifediol, calcitriol, carboplatin, carmustine, carprofen, cefaclor, cefixime, cefinetazole, cefonicid, cefoperazone, cefoperazone/sulbactam, cefotaxime, cefotetan, cefoxitin, ceftazidime, ceftezole, ceftibuten, ceftizoxime, ceftriaxone, cidofovir, cisplatin, clarithromycin, cloxacillin, cotrimoxazole, cyclosporine, dibekacin, doxorubicin hydrochloride liposome, doxycycline, ergocalciferol, erythromycin/sulfisoxazole, gentamicin, gold sodium thiomalate, ifosfamide, iohexol, isoniazid, kanamycin, lincomycin, lithium, lymphocyte immune globulin, meropenem, mesna, methicillin, mitomycin, metronidazole, nedaplatin, neomycin, netilmicin, oxaliplatin, paromomycin, peplomycin, phenylpropanolamine, penicillin G, piperacillin, pirarubicin, plicamycin, polymyxin B, praziquantel, rabbit antithymocyte globulin, recombinant human hemoglobin, rifampin, silver nitrate, streptomycin, teicoplanin, tetracycline, ticarcillin, tobramycin, and vancomycin.

In particular, the therapeutic agent is selected from the group consisting of aminoglycosides, such as gentamicin, kanamycin, neomycin, paramycin, ribostamycin, lividomycin, amikacin, dibekacin, butakacin, tobramycin, streptomycin, dihydrostreptomycin, sisomicin, verdamicin, nefilmicin, and butikacin, cisplatin, amphotericin B, ifosfamide, polymyxin B, cyclophosphomide, methotrexate, aprotinin, ciclosporin, and valproate as well as therapeutic antibodies. More preferably, the therapeutic agent is an aminoglycoside, such as gentamicin.

The compounds to be modified by the present invention are compounds having at least one cationic group and causing cell toxicity due to binding to megalin receptors leading to uptake of the compound, more preferably the therapeutic agent comprises a compound comprising at least two amino groups.

Once the compounds have been modified, the modified compound is preferably not able to bind to the megalin receptor, and thereby not able to be taken up by cells as assessed by the methods described herein and shown in the examples below.

Therapeutic Agent

The therapeutic agent according to the invention may be any therapeutic agent capable of causing organ damages due to intracellular accumulation in cells in the organs. In particular the therapeutic agent is capable of accumulating in cells in the kidneys and/or inner ear, thus causing kidney damages as well as damages to the inner ear.

Examples of therapeutic agents may be any of the following therapeutic agents:

acebutolol, acetazolamide, acyclovir, adefovir, albumin, alclofenac, alendronate, alitretinoin, altretamine, amikacin, amiloride, aminoglutethimide, amiodarone, amoxicillin, amoxicillin/clavulanic acid, amphotericin b, amphotericin b cholesteryl sulfate complex, amphotericin b lipid complex, amphotericin b liposome, amtolmetin, aniracetam, antacids, antazoline, anthraquinone laxatives, aprotinin, arginine, arsenic trioxide, asparaginase, aspirin, atenolol, atovaquone, auranofin, aurothioglucose, azacitidine, azathioprine, azlocillin, aztreonam, bacampicillin, bacitracin, bemetizide, benoxaprofen, betaine, bezafibrate, bismuth subcitrate, bleomycin, boric acid, brivudine, broxuridine, bumetanide, calcifediol, calcitriol, candesartan, candesartan/hydrochlorothiazide, canrenoate, capreomycin, captopril, carbenicillin, carboplatin, carmustine, carprofen, cefaclor, cefetamet, cefixime, cefinetazole, cefonicid, cefoperazone, cefoperazone/sulbactam, cefotaxime, cefotetan, cefoxitin, cefpirome, cefsulodin, ceftazidime, ceftezole, ceftibuten, ceftizoxime, ceftriaxone, cefuroxime, celecoxib, cephalexin, cephaloridine, cephalothin, cephapirin, cephradine, chlortetracycline, cidofovir, cilazapril, cimetidine, ciprofibrate, cisapride, cisplatin, clarithromycin, clodronate, clofibrate, cloxacillin, cocaine, codeine, colistin, corticotropin, cosyntropin, cotrimazine, cotrimoxazole, crisnatol, cyclacillin, cyclosporine, cysteamine, decitabine, delapril, delavirdine, demeclocycline, denileukin diftitox, desflurane, dextran, diatrizoate, diazoxide, dibekacin, diclofenac, diclofenac/misoprostol, dicloxacillin, dicumarol, didanosine, dihydroergotamine, dihydroergotamine/heparin, dihydrotachysterol, dirithromycin, dopamine, doxepin, doxorubicin hydrochloride liposome, doxycycline, edetate calcium disodium, edetate disodium, emetine, enflurane, enlimomab, epinephrine, epirubicin, ergocalciferol, ergotamine, erythromycin/sulfisoxazole, erythropoietins, ethanolamine oleate, ethyl chloride, etidronate, etodolac, etomidate, etretinate, everninomycin, fadrozole, fenbufen, fenofibrate, fenoprofen, fenoterol/ipratropium, flecainide, fleroxacin, floxacillin, flupirtine, flurbiprofen, formestane, foscarnet, fosinopril, fotemustine, framycetin, furosemide, gabexate, gadopentetate dimeglumine, gallium nitrate, gemcitabine, gemfibrozil, gentamicin, glycerin, gold sodium thiomalate, guanadrel, guanethidine, guar gum, halothane, hemiacidrin, hemin, hetastarch, homoharringtonine, hyaluronidase, hydrochlorothiazide, idarubicin, ifosfamide, imatinib, imipramine, indapamide, influenza vaccine, interferon alfa-2a, interferon alfa-2b, interferon beta, natural, interferon beta-1b, interferon gamma, interleukin-3, interleukin-4, interleukin-6, iobenguane I-131, iodixanol, iohexol, iopamidol, iopanoic acid, iopentol, iopromide, iotrolan, ioversol, ioxaglate, ioxilan, ioxithalamate, irinotecan, irofulven, isepamicin, isoflurane, isoniazid, isoxicam, kanamycin, ketamine, ketoconazole, ketoprofen, ketorolac, lenograstim, levofloxacin, lincomycin, liposomal nystatin, lisinopril, lithium, lobaplatin, lomustine, lonidamine, lornoxicam, losartan, loxapine, lymphocyte immune globulin, mannitol, mebendazole, mefenamic acid, meglumine antimoniate, melarsoprol, meropenem, mesna, metaraminol, methacycline, methicillin, methimazole, methocarbamol, methotrexate, methoxamine, metrizamide, metronidazole, meziocillin, milrinone, miltefosine, minocycline, minoxidil, mitoguazone, mitolactol, mitomycin, mitotane, molindone, momiflumate, morphine, moxalactam, muromonab-CD3, nabumetone, nafcillin, naproxen, nedaplatin, neomycin, netilmicin, niclosamide, nifedipine, niflumic acid, nifurtimox, nisoldipine, nitroprusside, norepinephrine, norfloxacin, ofloxacin, olsalazine, oxaliplatin, oxandrolone, oxaprozin, oxolinic acid, oxytetracycline, paclitaxel, pamidronate, paramethadione, paromomycin, pefloxacin, pemetrexed, pemirolast, penicillin G, pentamidine, pentostatin, peplomycin, perindopril, phenazopyridine, phenindione, phenobarbital, phenylbutazone, phenylpropanolamine, phenyloin, phosphates, piperacillin, pirarubicin, piretanide, piroxicam, plicamycin, poloxamer-188, polymyxin B, potassium perchlorate, praziquantel, proglumetacin, propylthiouracil, pyrimethamine/sulfadoxine, quinagolide, quinapril, quinine, rabbit antithymocyte globulin, raltitrexed, ranitidine, ranpirnase, recombinant human hemoglobin, rifampin, ritodrine, ritonavir, rofecoxib, rolitetracycline, rufloxacin, salsalate, sevoflurane, silver nitrate, silver sulfadiazine, simvastatin, sodium cellulose phosphate, sodium chloride, sodium fluoride, sodium stibogluconate, spironolactone, streptokinase, streptomycin, streptozocin, sulfamethoxazole, sulfasalazine, sulfinpyrazone, sulfisoxazole, sulindac, suiprostone, sultamicillin, suprofen, tacrolimus, tasonermin, teicoplanin, temafloxacin, teniposide, tenoxicam, tetracycline, thiopental, tiaprofenic acid, ticarcillin, ticrynafen, tiludronate, tiopronin, tobramycin, tocainide, tolazoline, tolmetin, torsemide, tramadol, triamterene, trimethadione, trimethaphan, trimethoprim, trimetrexate, trimipramine, troglitazone, tromethamine, typhoid vaccine, valsartan, vancomycin, zolimomab aritox, zomepirac, zopiclone.

Preferably, the therapeutic agent is selected from the group consisting of acyclovir, albumin, amikacin, amoxicillin, amoxicillin/clavulanic acid, amphotericin B, amphotericin B cholesteryl sulfate complex, amphotericin B lipid complex, amphotericin B liposome, aprotinin, arsenic trioxide, azathioprine, aziocillin, bacitracin, bismuth subcitrate, bleomycin, brivudine, calcifediol, calcitriol, carboplatin, carmustine, carprofen, cefaclor, cefixime, cefinetazole, cefonicid, cefoperazone, cefoperazone/sulbactam, cefotaxime, cefotetan, cefoxitin, ceftazidime, ceftezole, ceftibuten, ceftizoxime, ceftriaxone, cidofovir, cisplatin, clarithromycin, cloxacillin, cotrimoxazole, cyclosporine, dibekacin, doxorubicin hydrochloride liposome, doxycycline, ergocalciferol, erythromycin/sulfisoxazole, gentamicin, gold sodium thiomalate, ifosfamide, iohexol, isoniazid, kanamycin, lincomycin, lithium, lymphocyte immune globulin, meropenem, mesna, methicillin, mitomycin, metronidazole, nedaplatin, neomycin, netilmicin, oxaliplatin, paromomycin, peplomycin, phenylpropanolamine, penicillin G, piperacillin, pirarubicin, plicamycin, polymyxin B, praziquantel, rabbit antithymocyte globulin, recombinant human hemoglobin, rifampin, silver nitrate, streptomycin, teicoplanin, tetracycline, ticarcillin, tobramycin, and vancomycin.

In particular the therapeutic agent is selected from aminoglycosides, such as gentamicin, kanamycin, neomycin, paramycin, ribostamycin, lividomycin, amikacin, dibekacin, butakacin, tobramycin, streptomycin, dihydrostreptomycin, sisomicin, verdamicin, nefilmicin, and butikacin, cisplatin, amphotericin B, ifosfamide, polymyxin B, cyclophosphomide, methotrexate, aprotinin, ciclosporin, and valproate as well as therapeutic antibodies. In a preferred embodiment, the therapeutic agent is an aminoglycoside, such as gentamicin and kanamycin.

Also fusion proteins or fusion products used for medical treatment wherein one of the proteins is capable of binding the megalin or the receptor cubilin and/or a co-receptor of megalin and cubilin and the other protein/product causes cell toxicity when accumulating in the cells, may be used. In particular fusion products, wherein one part of the product is an antibody or IgG light chain, both capable of unspecifically binding to cubilin, and the other part of the product is cytotoxic, such as cancer treatment, may be co-administered with a compound according to the present invention in order to reduce organ damage, in particular kidney damage.

Dosages

The dosage of the compound according to the invention depends on the compound in question; however, the amount of the compound is also closely related to the therapeutic agent co-administered with the compound as well as the dosage of said therapeutic agent.

For all methods of use disclosed herein for the compounds, the daily oral dosage regimen will preferably be from about 0.01 to about 80 mg/kg of total body weight. The daily parenteral dosage regimen will be from about 0.001 to about 80 mg/kg of total body weight.

The term “unit dosage form” as used herein refers to physically discrete units suitable as unitary dosages for human and animal individuals, each unit containing a predetermined quantity of a compound, alone or in combination with other agents, calculated in an amount sufficient to produce the desired effect in association with a pharmaceutically acceptable diluent, carrier, or vehicle. The specifications for the unit dosage forms of the present invention depend on the particular compound or compounds employed and the effect to be achieved, as well as the pharmacodynamics associated with each compound in the host. The dose administered should be an “effective amount” or an amount necessary to achieve an “effective level” in the individual patient.

Since the “effective level” is used as the preferred endpoint for dosing, the actual dose and schedule can vary, depending on inter-individual differences in pharmacokinetics, drug distribution, and metabolism. The “effective level” can be defined, for example, as the blood or tissue level desired in the individual that corresponds to a concentration of one or more compounds according to the invention. Also, the effective level is depending on the therapeutic agent in question, and in particular on the concentration of the effective level in question.

Accordingly, in a preferred embodiment the ratio of the compound administered to the therapeutic agent administered is in the interval of from 200:1 mol:mol to 1:200 mol:mol, such as in the interval of from 100:1 mol:mol to 1:50 mol:mol, such as in the interval of from 50:1 mol:mol to 1:25 mol:mol

The compound may be administered in any suitable dosage regime, but is preferably administered with the same intervals as the therapeutic agent, preferably either shortly before or during administration of the therapeutic agent.

Most of the therapeutic agents according to this invention are administered parenterally, often intravenously. The compound according to the invention may be administered in any suitable manner according to the formulation thereof, it is however often preferred that the compound is administered parenterally, such as intravenously as the therapeutic agent.

Medicament

According to the invention the present medicament is capable of binding to the receptor megalin.

In a further embodiment the medicament is capable of binding to the receptor cubilin.

In yet a further embodiment the medicament is capable of binding to a co-receptor of megalin and cubilin.

In another embodiment the medicament is capable of binding to a therapeutic agent capable of binding to the receptor megalin and/or the receptor cubilin and/or a co-receptor of megalin and cubilin.

Combination Medicament

The present invention further relates to a combination medicament comprising the compound according to the invention in combination with a therapeutic agent. Thus, the invention further relates to a combination medicament comprising a compound as disclosed by the invention and a therapeutic agent for simultaneous, separate or sequential use in cell toxicity therapy.

In one embodiment said cell presents a receptor megalin and/or a receptor cubilin and/or a co-receptor of megalin and cubilin.

Administration

When using the present medicament the compound and the therapeutic agent may be administered simultaneously, either as separate formulations or combined in a unit dosage form, or administered sequentially. The combination medicament may be formulated by co-formulating the compound according to the invention with the therapeutic agent for simultaneous administration. In another embodiment the combination medicament is formulated as two separate medicaments for either simultaneous or sequential administration.

By the term “separate administration” is meant an initial administration of a first compound/medicament followed by secondary administration of a compound/medicament. The order of administration of the compounds/medicaments is not significant, and the time interval with which the first administration is followed by the second administration is not determined.

The main routes of drug delivery according to the present invention are intravenous, oral, and topical, as will be described below. Other drug administration methods, such as subcutaneous injection, which are effective to deliver the drug to a target site or to introduce the drug into the bloodstream, are also contemplated.

The mucosal membrane to which the pharmaceutical preparation of the invention is administered may be any mucosal membrane of the mammal to which the biologically active compound is to be given, e.g. in the nose, vagina, eye, mouth, genital tract, lungs, gastrointestinal tract, or rectum.

Compounds of the invention may be administered parenterally, that is by intravenous, intramuscular, subcutaneous, intranasal, intrarectal, intravaginal or intraperitoneal administration. The subcutaneous and intramuscular forms of parenteral administration are generally preferred. Appropriate dosage forms for such administration may be prepared by conventional techniques. The compounds may also be administered by inhalation, such as by intranasal and oral inhalation administration.

The compounds according to the invention may be administered with at least one other compound. The compounds may be administered simultaneously, either as separate formulations or combined in a unit dosage form, or administered sequentially. The combination medicament may be formulated by co-formulating the compound according to the invention with the therapeutic agent for simultaneous administration. In another embodiment the combination medicament is formulated as two separate medicaments for either simultaneous or sequential administration.

Pharmaceutical compositions containing a compound of the present invention may be prepared by conventional techniques, e.g. as described in Remington: The Science and Practice of Pharmacy 1995, edited by E. W. Martin, Mack Publishing Company, 19th edition, Easton, Pa. The compositions may appear in conventional forms, for example capsules, tablets, aerosols, solutions, suspensions or topical applications.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilising and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The parenteral formulations will typically contain from about 0.5 to about 25% by weight of the active ingredient in solution. Preservatives and buffers may be used. In order to minimise or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

### EXAMPLES

The following are non-limiting examples illustrating the invention.

### Example 1

The effect of a compound, 2-[4-(2-aminoethyl)piperazin-1-yl]ethylamine (Aldrich, D2,340-8, lot 06028BO-332), according to the invention was tested in vivo. Gentamicin uptake in mouse kidneys following intraperitoneal (i.p.) administration of the compound was measured.

For normal application of radioactive gentamicin the following was performed: I.p. injection of 50 micrograms per kg of tritiated gentamicin with or without 3 mg of the inhibitor per mouse. This equals 1% of the clinical dose of gentamicin used in patients.

For competition of clinical doses of gentamicin 50 micrograms/kg of tritiated gentamicin plus 5 mg/kg of cold gentamicin (clinical dose) were injected in admixture with 3 mg total of 2-[4-(2-aminoethyl)piperazin-1-yl]ethylamine per mouse. A control received gentamicin only. Inhibition was 40% compared to the control.

### Example 2

In Vitro Test

Gentamicin-inhibitor interactions were assessed by surface plasmon resonance (SPR) analysis on a Biacore 2000 instrument (Biacore, Uppsala, Sweden). Megalin purified from rabbit kidneys as described in Birn et al. (J. Biol. Chem., 1997, Vol. 272, No. 42, 26497-26504) was immobilized in a concentration of 28-40 fmol/mm². Samples were dissolved in 10 mM Hepes, 150 mM NaCl, 1.5 mM CaCl₂, 1 mM EGTA, 0.005% Tween-20 pH 7.4. The same buffer was used as running buffer. Regeneration of the sensor chip after each analysis cycle was performed with 1.6 M glycine-HCl buffer pH 3.0. The Biacore response is expressed in relative response units (RU) i.e. the difference in response between protein and control flow channel. Samples contained 2 mM gentamicin and 0-10 mM or 0-20 mM inhibitor. The response was read at maximum and corrected for contribution of the inhibitor response.

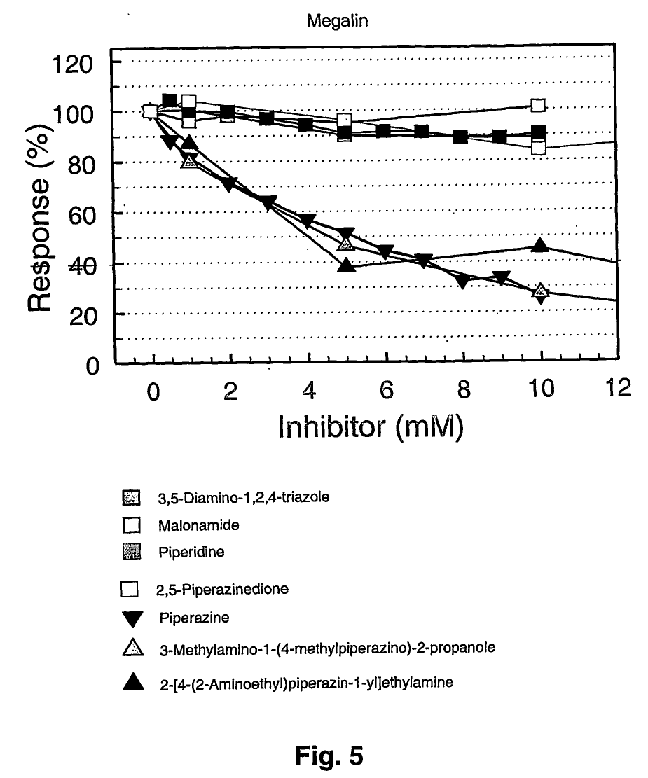

The experiment examines the inhibiting effect of 3,5-diamino-1,2,4-triazole, malonamide, piperidine, 2,5-piperazinedione, piperazine, 3-methylamino-1-(4-methylpiperazino)-2-propanole and 2-[4-(2-aminoethyl)piperazin-1-yl]ethylamine. The results are shown in FIG. 5.

### Example 3

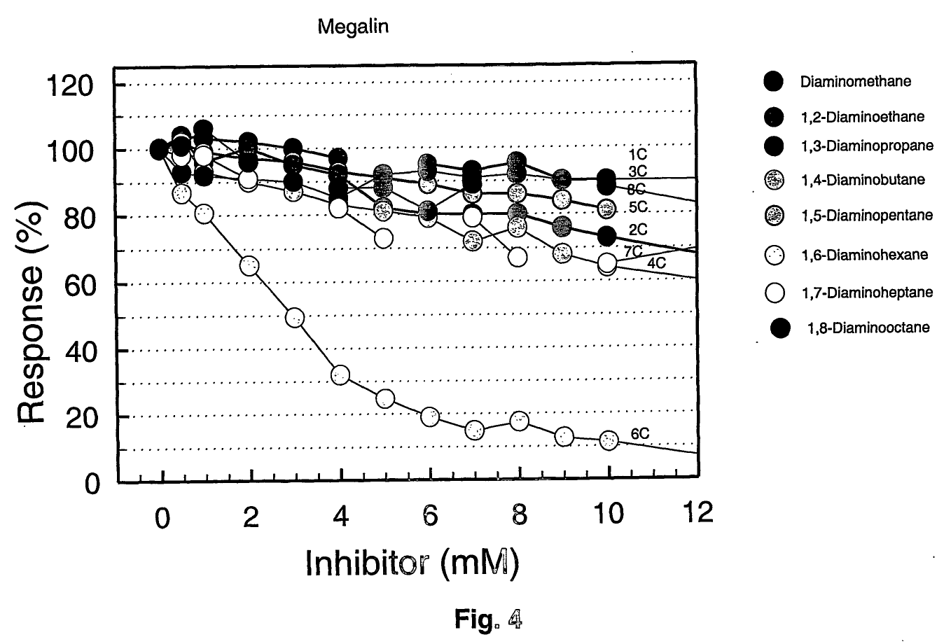

This experiment shows the inhibiting effect of diaminoalkanes by assays described in Example 2. The results are shown in FIG. 4.

### Example 4

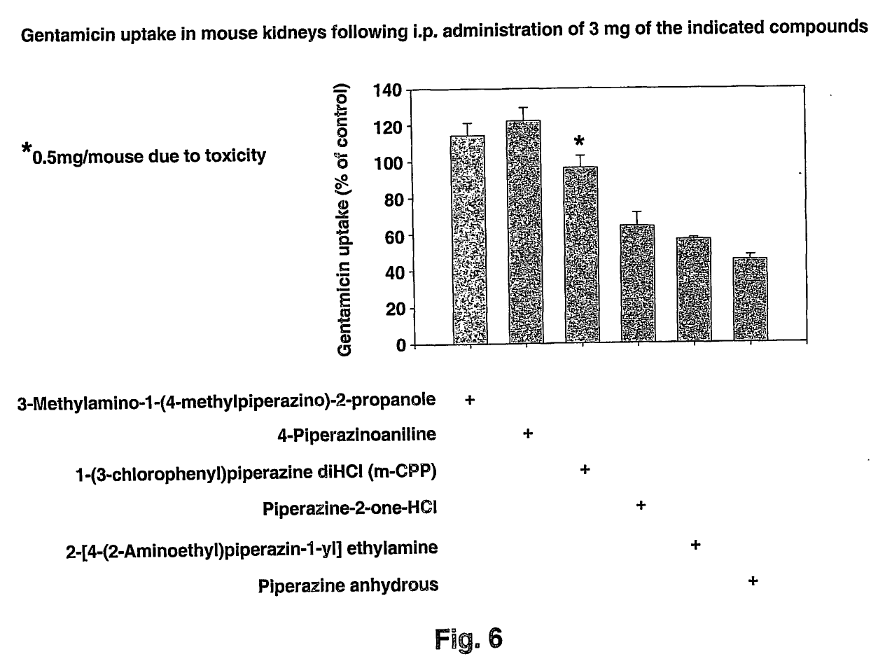

In this experiment 3 mg of the inhibitors 3-methylamino-1-(4-methylpiperazino)-2-propanole, 4-piperazinoaniline, piperazin-2-one-HCl, 2-[4-(2-aminoethyl)piperazin-1-yl]ethylamine, piperazine anhydrous, and 0.5 mg of 1-(3-chlorophenyl)piperazine diHCl (m-CPP), respectively, were administered intraperitoneally in mice (see FIG. 6) as described in Example 1. The effect of the inhibitors on the uptake of gentamicin into the mouse kidneys was determined by means of the assay described in Example 1. The strongest gentamicin inhibitors were piperazin-2-one-HCl, 2-[4-(2-aminoethyl)piperazin-1-yl]ethylamine, and piperazine anhydrous.

The reagents used in the Examples were the following:
2,4-diamino-6-phenyl-1,3,5-triazine: Catalogue: Aldrich; D2,340-8; lot: 06028BO-332
2-[4-(2-Aminoethyl)piperazin-1-yl]ethylamine: Catalogue: Interchim; 04386; lot: unknown
4-piperazinoaniline: Catalogue: Interchim; 04388; lot: unknown
3-Methylamino-1-(4-methylpiperazino)-2-propanol: Catalogue: Interchim; O4176; lot: unknown
Piperazine: Catalogue: Aldrich; P4,590-7; lot: 90907014
Piperidine: Catalogue: Aldrich; P4,610-5; lot: S11666-372
Piperazin-2-one: Catalogue: Aldrich; S99,763-3; lot: 0100945477
3,5-Diamino-1,2,4-triazole: Catalogue: Aldrich; D2,620-2; lot: S07917-042
Arginine: Catalogue: Aldrich; 13,846-0; lot: 01907C0
Malonamide: Catalogue: Aldrich; 12,959-3; lot: 3259397
1-(3-chlorophenyl)piperazine: Catalogue: Sigma; S014; lot: 048H4691
2,5-piperazinedione: Catalogue: Sigma; G6406; lot: 12420TI482
Diaminomethane: Catalogue: Fluka; 66770; lot: unknown
1,2-Diaminoethane: Catalogue: Sigma; E-2126; lot: 012K3463
1,3-Diaminopropane: Catalogue: Aldrich; D23807-5G; lot: 05516LQ
1,4-Diaminobutane: Catalogue: Aldrich; 234001-25G; lot: 10203TS
1,5-Diaminopentane: Catalogue: Aldrich; 27,182-9; lot: 08208P0
1,6-Diaminohexane: Catalogue: Sigma; H-2381; lot: 12K3452
1,7-Diaminoheptane: Catalogue: Sigma; D-3266; lot: 81K3670
1,8-Diaminooctane: Catalogue: Aldrich, D2,240-1; lot: 33501-030

1-61. (canceled)

62. A method for the prophylaxis and/or treatment of induced cell toxicity, comprising the step of administering a compound comprising a structure of the general formula (VI) 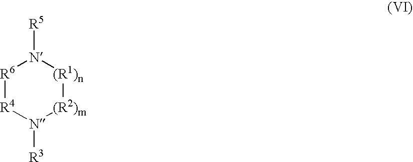
wherein
each R¹ and each R² independently are selected from C, S, N, O, optionally substituted with C, S, N, O, OH, hydrogen, alkyl, alkenyl, alkynyl, phenyl, benzyl, amine (NH), halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, optionally further substituted one or more times with C, S, N, O, OH, phenyl, amine (NH), halogen, substituted lower alkyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, halogen, trifluoromethyl, cyano, amino, or nitro, and wherein
m is an integer of from 1 to 8,
n is an integer of from 1 to 8,
N′ and N″ are nitrogen,
R³, R⁴, R and R⁶ are independently selected from C, S, N, O, OH, hydrogen, alkyl, alkenyl, alkynyl, phenyl, benzyl, amine (NH), halogen, substituted lower alkyl, aryl, heterocycloalkyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, optionally further substituted one or more times with C, S, N, O, OH, phenyl, amine (NH), halogen, substituted lower alkyl, aryl, heterocyclyl, heteroaryl, aryl-(C₁₋₄)-alkyl, heteroaryl-(C₁₋₄)-alkyl, heterocyclyl-(C₁₋₄)-alkyl, cycloalkylalkyl, cycloalkyl, alkoxy, carboxy, halogen, trifluoromethyl, cyano, amino, or nitro, or one or more of R³, R⁴, R⁵ and R⁶ is a chemical bond,
or a pharmaceutically acceptable addition salt or hydrate thereof,
or diaminomethane, 1,2-diaminoethane, 1,3-diaminopropane, 1,4-diaminobutane, 1,5-diaminopentane, 1,6-diaminohexane, 1,7-diaminoheptane, 1,8-diaminooctane,
or a pharmaceutically acceptable addition salt or hydrate thereof.

63. The method according to claim 62, wherein said cell presents the receptor megalin and/or the receptor cubilin.

64. The method according to claim 62, wherein at least one of R¹ or R² is C.

65. The method according to claim 62, wherein R¹ and R² are C.

66. The method according to claim 62, wherein at least one of R¹ or R² is S.

67. The method according to claim 62, wherein R¹ and R² are S.

68. The method according to claim 62, wherein at least one of R¹ and R² is N.

69. The method according to claim 62, wherein
R¹ and R² are N.

70. The method according to claim 62, wherein at least one of R¹ and R² is O.

71. The method according to claim 62, wherein R¹ and R² are O.

72. The method according to claim 62, wherein the medicament is capable of binding to the receptor megalin and/or the receptor cubilin.

73. The method according to claim 62, wherein the compound is selected from the group consisting of 3-methylamino-1-(4-methylpiperazino)-2-propanole, 4-piperazinoaniline, 1-(3-chlorophenyl)piperazine diHCl (m-CPP), piperazin-2-one-HCl, 2-[4-(2-aminoethyl)piperazin-1-yl]ethylamine, piperazine anhydrous, 2,4-diamino-6-phenyl-1,3,5-triazine, 3,5-diamino-1,2,4-triazole, 2,5-piperazinedione, piperazine, and piperazin-2-one-HCl, 1(2-pyrimidyl)piperazine dihydrochloride, or is a pharmaceutically acceptable addition salt or hydrate thereof.

74. The method according to claim 73, wherein the compound is selected from 2-[4-(2-aminoethyl)piperazin-1-yl]ethylamine, 3-methylamino-1(4-methylpiperazino)-2-propanole, and piperazine.

75. The method according to claim 62, wherein the compound is piperazine.

76. The method according to claim 62, wherein the compound is selected from the group consisting of 1,7-diaminoheptane, 1,2-diaminoethane, 1,4-diaminobutane, 1,6-diaminohexane, and 1,5-diaminopentane.

77. The method according to claim 62, wherein the compound is 1,6-diaminohexane.

78. The method according to claim 62, wherein the cell is from the kidney and/or the inner ear.

79. The method according to claim 62, wherein said compound in solution has at least one positive charge.

80. The method according to claim 62, wherein said compound has a polybasic charge distribution.

81. A compound having the general formula of
(A_{q}-X)ₚ (VII)
wherein
A is a compound as defined in claim 62, and wherein
X is a spacer,
q is an integer of 1-100,
p is an integer of 1-100.

82. The compound according to claim 81, wherein the spacer is a covalent bond.

83. The compound according to claim 81, wherein the spacer consists of from 2-12 atoms.

84. Method for prophylaxis and/or treatment of induced cell toxicity comprising the step of administering a compound as defined in claim 81.

85. A combination medicament comprising a compound as defined in claim 62 and a therapeutic agent for stimultaneous, separate or sequential use in induced cell toxicity therapy.

86. The combination medicament according to claim 85, wherein said cell presents the receptor megalin and/or the receptor cubilin.

87. A pharmaceutical composition comprising a compound as defined in claim 81 and pharmaceutically acceptable carriers, excipients or diluents therefor.
